(19) United States
(12) Patent Application Publication
(10) Pub. No.: US 2006/0160235 A1
(43) Pub. Date: Jul. 20, 2006

(54) Separation of denatured proteins in free solution

(76) Inventors: Harold G. Craighead, Ithaca, NY (US); Chang Lu, West Lafayette, IN (US)

Correspondence Address: Schwegman, Lundberg, Woessner & Kluth, P.A., P.O. Box 2938, Minneapolis, MN, 55402, US

(21) Appl. No.: 11/281,249

(22) Filed: Nov. 17, 2005

Related U.S. Application Data
(60) Provisional application No. 60/629,586, filed on Nov. 19, 2004.

(51) Int. Cl. G01N 33/00; C07K 1/10

(52) U.S. Cl. 436/86; 530/412

(57) ABSTRACT

The present invention relates generally to the separation of proteins in free solution using electrophoresis. The invention provides gel-free methods of separating proteins that involve the use of a mixture of alkyl sulfates of different carbon chain lengths.

### CROSS REFERENCE TO RELATED APPLICATIONS

This application claims priority from U.S. Provisional Application Ser. No. 60/629,586, filed Nov. 19, 2004.

### STATEMENT REGARDING FEDERALLY SPONSORED RESEARCH

Work relating to this application was supported by a grant from the U.S. Government (NSF-ECS-9876771). The government may have certain rights in the invention.

### TECHNICAL FIELD

The invention relates to methods of separating proteins in free solution.

### BACKGROUND

Electrophoresis of polyelectrolytes, such as DNA and denatured proteins, is usually performed in chemical or physical gels, except special cases. See e.g., Noolandi, Electrophoresis 13: 394 (1992); Han & Craighead, Science 288: 1026 (2000). In recent years, there has been a move towards miniaturization of gel electrophoresis as these methods offer the advantages of fast analysis time, automation, on-line data acquisition and storage capability and the requirement for smaller samples. See Khaledi et al., Chemical Analysis Series, Vol. 146, Chapter 5, John Wiley & Sons, InC. (1998); Guttman, Electrophoresis 17: 1333-1341(1996). However, gel-based electrophoresis in microchannels can be a challenge due to the difficulty of loading high-viscosity polymeric sieving matrices into microchannels. Furthermore, in free solution, proteins with molecular weights more than 10 kDa generally have the same electrophoretic mobilities after they are fully denatured by sodium dodecyl sulfate (SDS) and a reducing agent. Shirahama et al., J. Biochem. 75: 309 (1974); Karim et al., Electrophoresis 15:1141(1994). This phenomenon is attributed to the constant charge density along the polypeptide chain. The coating of the negatively charged surfactant makes the intrinsic charge of the proteins insignificant.

Thus, there is a need for alternate methods for separating polyelectrolytes such as nucleic acids and denatured proteins.

### SUMMARY OF THE INVENTION

The invention provides novel methods of separating denatured proteins that do not require the use of chemical or physical gels. These methods make possible the separation of denatured proteins that are greater than 10 kDa in free solution. The free-solution methods of separating proteins provided by the invention have benefits associated with gel-free systems that include greater compatibility with other protein analysis techniques such as mass spectrometry. In addition, issues related to disruption of gel structures and the sensitivity of gels to temperature, ionic strength, pressure are avoided.

The invention involves the discovery that proteins that have formed complexes with alkyls of different carbon chain lengths have different electrophoretic mobilities in free solution and thus may be separated. Differences in electrophoretic mobilities stem from differential binding of alkyl sulfates with different carbon chain lengths to the protein surface. The binding of different alkyl sulfates to a given protein is determined by hydrophobicities of the amino acids residues in the protein sequence. Accordingly, the present invention provides methods for separating denatured proteins in free solution.

In one embodiment, the invention provides a method for separating proteins that involve contacting the proteins with a mixture of alkyl sulfates having different carbon chain lengths and separating the proteins in free solution using electrophoresis. At least one of the proteins that may be separated according to the methods of the invention may be greater than about 10 kDa. Methods of the invention may also be used to separate labeled proteins. The proteins may be labeled with a fluorogenic dye or with a radioactive label such as S³⁵. The mixture of alkyl sulfates may include an alkyl sulfate selected from the group consisting of dodecyl sulfate, tetradecyl sulfate, and hexadecyl sulfate. In another embodiment, the mixture of alkyl sulfates may have alkyl sulfates with about two, three, four or five different carbon chain lengths. The mixture of alkyl sulfates may have two or three of the following: dodecyl sulfate, tetradecyl sulfate, and hexadecyl sulfate.

Methods of the invention involve denaturing proteins in the presence of a mixture of alkyl sulfates having different carbon chain lengths and separating the proteins using free solution electrophoresis. Proteins may be denatured using heat. For example, proteins may be denatured by heating to about 95° C. for about 5 minutes in the presence of alkyl sulfates. Proteins may also be denatured by heating to about 80° C. to about 95° C. for about 10 minutes or to about 50° C. to about 80° C. for about 30 minutes in the presence of alkyl sulfates.

According to the methods of the invention, proteins may be in contact with a mixture of alkyl sulfates having different carbon chain lengths during separation. The alkyl sulfates may have about two, three, four or five different carbon chain lengths. Two or three of the alkyl sulfates may be the following: dodecyl sulfate, tetradecyl sulfate, and hexadecyl sulfate. The separation may be performed on a microfluidic chip or in a capillary tube.

In another embodiment, the methods of the invention may involve detecting at least one of the separated proteins. Proteins may be detected using laser-induced fluorescence.

In another embodiment, the methods of the invention may involve isolating at least one of the separated proteins. For example, the invention provides a method for isolating proteins that involves: (a) obtaining a biological sample that comprises proteins; (b) denaturing the proteins while they are in contact with a mixture of alkyl sulfates having different carbon chain lengths; and (c) separating the proteins using electrophoresis while they are in contact with a mixture of alkyl sulfates having different carbon chain lengths. Proteins that may be isolated according to the methods of the invention may also be labeled with a detectable marker.

In another embodiment, the invention provides a composition of one or more purified proteins and a mixture of alkyl sulfates having different carbon chain lengths. As used herein, the term “purified” in reference to a protein refers to a protein that has been separated from components which naturally accompany it. A protein is usually considered to be pure when it is at least 60%, preferably at least 75%, more preferably at least 90%, and most preferably at least 99%, by weight, free from other proteins and naturally-occurring organic molecules with which it is naturally associated. A purified protein may be obtained, for example, by extraction from a natural source such as a cell; by expression of a recombinant nucleic acid encoding the protein; or by chemically synthesizing the protein. Purity may be measured by any appropriate method such as, for example, column chromatography, polyacrylamide gel electrophoresis, or high performance liquid chromatography.

The composition may include about 2 to about 10 different purified proteins, and at least one of the purified proteins of the composition may be greater than 10 kDa. In addition, the composition has alkyl sulfates of about two, three or four different carbon chain lengths; the different carbon chain lengths may be from about three to about eighteen carbons. The composition may include dodecyl sulfate, tetradecyl sulfate, and hexadecyl sulfate or any combinations of these.

The present invention also provides kits and apparatus for performing certain methods of the present invention.

According to the methods of the invention, a mixture of alkyl sulfates with different carbon chain lengths may be used in the sample buffer and running buffer for protein denaturation and electrophoresis. Under this treatment, the electrophoretic mobilities of denatured proteins are very different in free solution. Although it is not meant as a limitation of the invention, it is believed that differences in the electrophoretic mobility among various denatured proteins are attributed to the heterogeneous binding of mixed alkyl sulfates to the protein surface, which is characteristic of the amino acid sequence. The coating of the surfactants determines the effective size and the charge-to-mass ratio of the protein-detergent complex, and therefore determines the mobility. Thus, the methods of the invention allows for protein separation based on amino acid sequence, in particular, on the characteristic distribution of hydrophobic amino acid residues. In addition, according to the methods of the invention, free solution electrophoresis may be performed on a glass microchip or using a conventional capillary tube. These methods avoid the difficulties associated with gels when used as a one-dimensional tool. In addition, since the separation is based on amino-acid sequence and independent of the molecular weight, methods of the invention can be coupled with gel electrophoresis to form a two-dimensional system. Thus, methods of the invention may be coupled to SDS-PAGE.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### BRIEF DESCRIPTION OF THE FIGURES

FIG. 1 is a graphical illustration of the free-solution microchip electrophoretic separation of four proteins, α-lactalbumin, ovalbumin, conalbumin, and β-galactosidase, after denaturation in the presence of Pierce sodium dodecyl sulfate (SDS).

FIG. 2 is a comparison of the free-solution electrophoretic separation of four proteins, α-lactalbumin, ovalbumin, conalbumin, and β-galactosidase, after denaturation in the presence of SDS, sodium tetradecyl sulfate (STS), or a binary mixture of SDS/STS (3:1, w/w).

FIG. 3 is a schematic illustration of the differential binding of SDS and STS to the protein surface and the effect of the amino acid sequence on binding. The longer surfactant is STS. The different color spheres represent amino acid residue clusters with different hydrophobicities. The drawing does not reflect the actual packing density of the surfactants.

FIG. 4 is a schematic illustration of the layout of the microchip used in free-solution electrophoresis.

FIG. 5 is an illustration of the reproducibility of eleven consecutive experiments in which mixtures of β-galactosidase, conalbumin, α-lactalbumin, and ovalbumin were denatured and subjected to free solution microchip electrophoretic separation.

### DETAILED DESCRIPTION OF THE INVENTION

The invention provides novel methods of separating denatured proteins that do not require the use of chemical or physical gels. These methods make possible the separation of denatured proteins that are greater than 10 kDa in free solution. The free-solution methods of separating proteins provided by the invention have benefits associated with gel-free systems that include greater compatibility with other protein analysis techniques such as mass spectrometry. In addition, issues related to disruption of gel structures and the sensitivity of gels to temperature, ionic strength, pressure are avoided. Thus, the invention features methods for separating proteins in free solution, that is, without the use of chemical or physical gels.

The invention involves the discovery that proteins that have formed complexes with alkyl sulfates of different carbon chain lengths have different electrophoretic mobility in free solution and thus may be separated in free solution using electrophoresis. Differences in electrophoretic mobility stem from differential binding of alkyl sulfates with different carbon chain lengths to the protein surface. The binding of alkyl sulfates to a given protein sequence is determined by hydrophobicities of the amino acids residues in the protein sequence.

Proteins

Proteins that may be separated using the methods of the invention include polymers of natural or non-natural amino acid residues linked by peptide bonds. Proteins also may be any lengths and may have any post-translational modifications such as glycosylation or phosphorylation.

Proteins that may be separated using the methods of the invention may be from any source. For example, proteins may be from a biological source such as bacteria, plant, animal and human. The biological source may be a cell or tissue that naturally expresses the protein as well as a cell, cell culture or tissue that has been engineered to express the protein using recombinant DNA methodologies. Thus, protein samples that may be separated using the methods of the invention include the supernatant of a cell culture, a cell lysate or homogenate, a partially-purified protein preparation, as well as blood, urine, and other bodily fluids. Conventional methods that may be used in obtaining these samples include centrifugation, homogenization, French press, the use of lysozymes, osmotic shock and the like, as well as collection methods commonly used for bodily fluids. Protein samples that may be separated using the methods of the invention also include proteins generated by a synthetic chemical reaction such as, for example, the reaction described in Merrifield et al., (1956) J. Am. Chem. Soc. 78: 4646 and Merrifield (1963) J. Am. Chem. Soc. 85:2149.

The term protein includes proteins in their native forms as well as denatured proteins. Denatured proteins lose their three-dimensional folded structure, that is, they lose their characteristic secondary, tertiary and quaternary structures. Proteins may be denatured using reducing agents such as 2-mercaptoethanol. Proteins may be denatured in the presence of alkyl sulfates having different carbon chain lengths. For example, proteins may be denatured in the presence of Pierce SDS or in the presence of mixtures of two, three or four long chain alkyl sulfates including pure dodecyl sulfate, pure tetradecyl sulfate or hexadecyl sulfate. Proteins may also be denatured by heating. For example, proteins may be combined with alkyl sulfates having different carbon chain lengths and the resulting sample heated for an appropriate period of time at a selected temperature. Thus, proteins may be denatured by heating to 95° C. for 5 minutes or by heating at a lower temperature for longer periods of time. Methods of denaturing proteins are known in the art.

Proteins may be unlabeled or labeled. Proteins that are unlabeled may be detected, for example, by UV absorbance. If labeled, a fluorogenic dye, for example, may be used. Examples of fluorogenic dyes include, without limitation, ATTO-TAG CBQCA (3-(4-carboxybenzoyl)quinoline-2-carboxaldehyde or CBQCA) and ATTO-TAG FQ (3-(2-furoyl)quinoline-2-carboxaldehyde) (Molecular Probes, Eugene, Oreg.). See, for example, http://probes.invitrogen.com/media/pis/mp02333.pdf (retrieved on Nov. 11, 2005) for additional information on these dyes. Proteins may also be radioactively labeled using, for example, S³⁵ or I¹²⁵. Proteins may be labeled using methods known in the art or methods provided by various manufacturers of protein labeling reagents or dyes. A protein or a sample comprising multiple proteins is said to be labeled when a sufficient proportion of the protein molecules is labeled such that the protein or different proteins may be detected using the appropriate detection method. For example, depending on the label being used, a sample may be labeled when 90%, 80%, 70% 60%, 50%, 40%, 30%, 20% 10% or even less than 10% of the proteins in the sample have the label.

Surfactants

A surfactant relates to an organic compound that contain a hydrophobic region such as, for example, a nonpolar hydrocarbon group and hydrophilic reagion such as, for example, a polar ionic region that may be negatively (anionic) or positively (cationic) charged. Surfactants useful for practicing the methods of the invention include anionic surfactants. Non-limiting examples of anionic surfactants include those which are based on sulfonate anions or carboxylate anions such as soaps or fatty acid salts, e.g. sodium and potassium salts of long-chain carboxylic acids; sodium dodecyl sulfate (SDS), ammonium lauryl sulfate, and other alkyl sulfate salts; and sodium laureth sulfate. For more information on anionic surfactants, see, for example, http://en.wikipedia.org/wiki/Surfactant (last retrieved Nov. 14, 2005); see also Streitwieser & Heathcock, Introduction to Organic Chemistry, 3^{rd} Edition, p. 456-458, 771, Macmillan Publishing Company, 1985; Mathews & van Holde, Biochemistry, p. 298-301, The Benjamin/Cummings Publishing Company, Inc., 1990.

Anionic surfactants useful for practicing methods of the invention include, for example, alkyl sulfates. Alkyls refer to linear or branched hydrocarbons, for example, a C₁-C₁₈ hydrocarbon, containing normal, secondary, tertiary or cyclic carbon atoms. Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃))₃. Alkyls useful for practicing the methods of the invention include alkyls having 1 to 18 carbons in length, preferably alkyls having 8 to 18 carbons. For example, alkyls useful for practicing the methods of the invention include alkyl sulfates such as sodium dodecyl sulfate (SDS), sodium tetradecyl sulfate (STS) and sodium hexadecyl sulfate (SHS). Alkyl sulfates useful for practicing the invention may be a mixture of two, three or four pure alkyl sulfates of different carbon chain lengths, such as for example, pure dodecyl sulfate, pure tetradecyl sulfate and pure hexadecyl sulfate. Alternatively, alkyl sulfates having more than four different carbon chain lengths, for example, Pierce SDS, which contains C₁₂, C₁₄, C₁₆, and C₁₀+C₁₈ may also be used.

Protein Separation in Free Solution

The invention provides methods of separating proteins in free solution using electrophoresis. The separation of proteins in free solution refers the separation of proteins in free solution, that is, without the use of a chemical or physical gel which functions as a sieving matrix. As such, proteins may be separated in a direct current field, that is, by electrophoresis. According to the methods of the present invention, protein separation may be performed on a microfluidic chip. A microfluidic chip may be made of glass, silica, or plastic. The microfluid chip may have narrow channels into which samples to be analyzed are injected. Protein separation may also be performed in a conventional capillary tubes. For more information on the use of capillary tubes, see, for example, High-performance Capillary Electrophoresis: Theory, Techniques, and Applications, ed. M. G. Khaledi, Chemical Analysis series, John Wiley & sons Inc, 1998, vol 146.

The methods of separating proteins in free solution, as provided by the invention, may be used in conjunction with gel electrophoresis and/or isoelectric focusing in a multidimensional separation system. The separation mechanism discovered in the invention is orthogonal to other methods such as gel electrophoresis and isoelectric focusing. Thus, the methods of the invention may be coupled with existing methods to enhance the separation efficiency. (Moore and Jorgenson, J. W. Anal. Chem. 1995, 67, 3456-3463).

Henry has derived the electrophoretic mobility of an infinitely long, charged, insulating cylinder in an aqueous salt solution (see Proc. R. Soc. London, Ser. A 133:106 (1931)). He showed that the mobility could be described with the equation (4)

$$μ = \frac{{ɛ}_{b} {ɛ}_{0} ζ}{4 π η} \qquad ( 4 )$$

Where ε_{b} is the dielectric constant, ε₀ is the permittivity of vacuum, ζ is the surface potential of the charged cylinder, and η is the viscosity of the solution. Stigter showed that in the case of a charged, insulting cylinder of finite length L and radius a, L/a>>1, while the radius a is of the same order as Debye length 1/κ, equation (4) was still a good approximation (see J. Phys. Chem. 82:1424 (1978)). Debye length 1/κ is given by

$${κ}^{- 1} = {( \frac{{ɛ}_{b} {ɛ}_{0} kT}{{ⅇ}^{2} {\sum }_{k} {z}_{i}^{2} {C}_{i}} )}^{1 / 2} \qquad ( 5 )$$

where k is the Boltzmann constant, T is the Kelvin temperature, e is the electron charge, and Cᵢ and Zᵢ are the concentration and charge valence of species i, respectively. Using Debye-Hückel approximation, the surface potential ζ can be related to the surface charge density σ.

$$ζ = \frac{σ}{{ɛ}_{b} {ɛ}_{0} κ} \qquad ( 6 )$$

Combining equations (4) and (6), provides

$$μ = \frac{σ}{4 π ηκ} \qquad ( 7 )$$

Equation (7) is by no means a rigorous theoretical treatment. However, it gives important suggestions about how to interpret the results presented herein. According to this result, the difference in the surface charge density is the main reason for different free solution mobilities of denatured proteins. In the examples presented herein, the packing density of the surfactants determines the surface charge density.

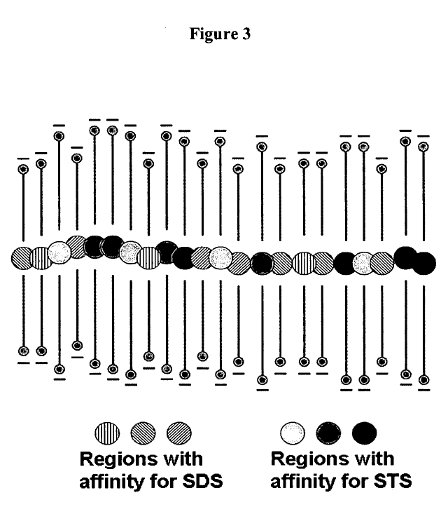

Thus, the separation of denatured proteins in free solution is thought to be due to the differential binding of alkyl sulfates with different carbon chain lengths on the polypeptide surface (FIG. 3). Longer-chained alkyl sulfates such as STS and SHS have higher hydrophobicity than SDS. Some amino acid residues are more hydrophobic than the others. Thus, the incorporation of alkyl sulfates with mixed chain lengths into the protein-sulfate complex appears to generate heterogeneity in the surface coating, which is determined by the amino acid sequence. The specific distribution of different alkyl sulfates is unique to each protein and determines the packing density of surfactants, and therefore, the free solution mobility. From the data presented herein, the free solution mobilites of the proteins denatured by the binary mixture SDS/STS were different from those of the proteins denatured by either pure SDS or pure STS. This indicates that the molecular packing density in the mixed coating is significantly different from that of a single surfactant coating. This novel mechanism provides a fast and simple method for protein analysis.

The invention will be further described in the following examples, which do not limit the scope of the invention described in the claims.

### EXAMPLES

The following examples describe the separation of four proteins in a microfluidic chip. The same or similar protocols may be implemented in conventional capillary tubes. (High-performance Capillary Electrophoresis: Theory, Techniques, and Applications, ed. M. G. Khaledi, Chemical Analysis series, John Wiley & sons Inc, 1998, vol 146.)

### Example 1

### Preparation of Proteins for Free Solution Electrophoresis

Surfactants with different compositions were used for the denaturation of proteins. The surfactants used in these experiments included: (1) Pierce SDS, a commercial alkyl sulfate mixture with multiple chain lengths available from Pierce (Rockford, Ill.; No. 28364, lot analysis: C₁₂ 73%, C₁₄ 24%, C₁₆ 3%, and C₁₀+C₁₈<1% w/w); (2) SDS (99%) from Sigma (St. Louis, Mo.); (3) STS (99%) from Lancaster Synthesis (Pelham, N.H.); and (4) SDS/STS, a binary mixture of SDS (99%) and STS (99%) (w/w=3:1).

The sample buffer for denaturation was 2.5 mM sodium borate (pH˜9.2) containing 1% 2-mercaptoethanol. In experiments involving Pierce SDS, pure SDS, and SDS/STS, the sample buffer contained 1.45% (w/v) surfactant. In experiments involving STS, the sample buffer contained 0.59% (w/v) STS due to its low solubility. Protein mixtures were denatured by heating in sample buffer at 95° C. for 5 minutes. Denaturation resulted in suppression of the heterogeneity of multiple labeling as the negatively charged detergent coating overwhelmed the intrinsic charge of the proteins and the charge of the dye molecules.

After denaturation, proteins were labeled using the fluorogenic dye FQ (3-(2-furoyl)quinoline-2-carboxaldehyde) from Molecular Probes (Eugene, Oreg.). The labeling procedure was similar to protocols described in the literature, e.g. Pinto et al., Anal. Chem. 69:3015 (1997); Hu et al., Electrophoresis 22:3677 (2001).

### Example 2

### Free Solution Microchip Electrophoresis with Laser-Induced Fluorescence Detection

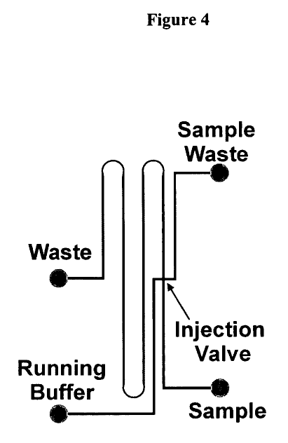

Microchips were fabricated on Pyrex 7740 wafers using photolithography, plasma etching, and thermal bonding techniques. FIG. 4 is an illustration of the design of the microchip. The microchannels had a depth of 3.8 μm and a half-depth width of 26 μm. The serpentine separation channel had a total length of 12 cm. The channel width at the turns was one half of the straight channel. The structure was previously reported for the minimization of geometric dispersion of analyte bands (see Molho et al., Anal. Chem. 73:1350 (2001); Ramsey et al., Anal. Chem. 75:3758 (2003)). The microfluidic chip was used with uncoated channel walls. Before the first use, the microfluidic chip was conditioned by flowing water and then 0.1 M NaOH for 20 minutes each. The running buffer was flowed for 20 minutes each time before the labeled-protein solution was loaded. After the experiment, the microchip was cleaned by running water, 0.1 M NaOH and then water again, each for 20 minutes. The microchip was stored with water in the channel and the reservoirs sealed with parafilm.

The running buffer for microchip electrophoresis consisted of 2.5 mM sodium borate and 5 mM of the surfactant or surfactant mixture used in the denaturation step. A low concentration of surfactant was used to maintain the coating on the protein surface during electrophoresis, and the concentration was below the critical micelle concentration (CMC). See Khaledi, Chemical Analysis Series, Vol. 146, p. 77-131, John Wiley & Sons InC. (1998).

Proteins were denatured and labeled according to the protocol described herein. A plug of labeled protein mixture was injected at the cross of the microfluidic channels using a gated injection scheme previously described (see Jacobson et al., Anal. Chem. 66: 3472 (1994); Ermakov et al., Anal. Chem. 72: 3512 (2000)).

Protein bands were detected with laser-induced fluorescence. An inverted microscope, Olympus, IX 70, was used in all experiments. An air-cooled argon ion laser provided the excitation at 488 nm. Fluorescence was collected by a 60×, 0.70 NA microscope objective, filtered with a 615DF45 bandpass filter (Omega Optical, Brattleboro, Vt.) to remove scattered light. The light was imaged onto a photomultiplier tube (R1477, Hamamatsu, Bridgewater, N.J.) biased at 1.2 kV. The resulting signal was amplified by a low-noise preamplifier (SR 560, Stanford Research Systems, Sunnyvale, Calif.) and digitized by an I/O board (PCI-MIO-16E-4, National Instruments). Software written in LabVIEW controlled the injection time and plotted the data.

### Example 3

### Protein Separation Using Microchip Electrophoresis in Free Solution with Laser-Induced Fluorescence Detection

These examples illustrate the separation of denatured proteins using microfluidic chip electrophoresis in free solution. Electrophoresis was performed after proteins were denatured with a mixture of alkyl sulfates with different carbon chain lengths.

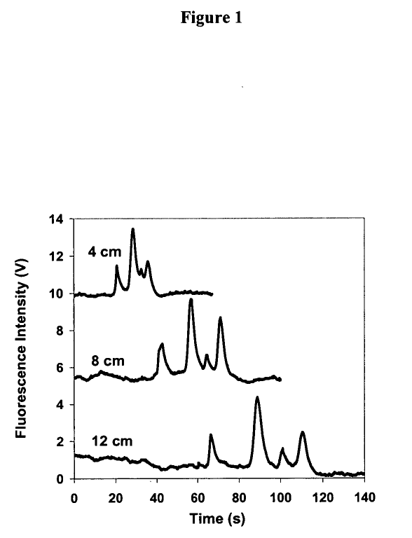

In one experiment, proteins were denatured using Pierce SDS and then separated using microchip electrophoresis in free solution. The running buffer consisted of 2.5 mM sodium borate+0.14% (w/v) Pierce SDS. The protein mixture consisted of about 1×10⁻⁶ M of each of four proteins: α-lactalbumin (14 kDa), ovalbumin (45 kDa), conalbumin (78 kDa), and β-galactosidase (116 kDa). Separation was conducted under an electric field of 436 V/cm in the separation channel, and fluorescence was detected at 4, 8, and 12 cm from the injection. Results are shown in FIG. 1. A single peak was observed for each denatured protein. The peaks were assigned as follows (12 cm): β-galactosidase—67 s; conalbumin—89 s; α-lactalbumin—101 s; and ovalbumin—111 s. The peaks were assigned by the comigration of a single protein with the mixture. At 436 V/cm in the separation channel and 12 cm from the injection point, the four denatured proteins were completely separated in free solution. The elution sequence of the proteins was not correlated with the molecular weights of the proteins, and thus, separation was not based on differences in molecular sizes.

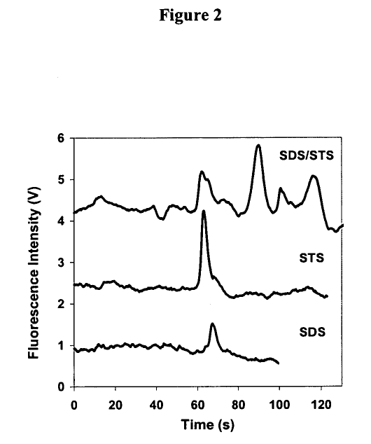

In a second experiment, proteins were denatured using pure SDS, pure STS, or using a binary mixture of SDS/STS (3:1, w/w), and then separated using microchip electrophoresis in free solution. The running buffer contained 2.5 mM sodium borate and 0.14% (w/v) surfactant when SDS or SDS/STS was used. When STS was used as the surfactant, however, the running buffer contained 0.04% (w/v) STS. The ratio of SDS to STS in the binary mixture was similar to what was in Pierce SDS. Protein separation was driven by an electrical field of 436 V/cm, and fluorescence was detected at 12 cm from the injection. Results are shown in FIG. 2. Proteins denatured by single component sulfate such as pure SDS or pure STS could not be separated in free solution electrophoresis. In contrast, proteins denatured using the binary mixture of pure SDS/STS could be separated in free solution electrophoresis. Thus, protein separation in experiments utilizing Pierce SDS did not stem from any minor impurity in the Pierce SDS. The results presented herein indicate that STS can also form STS-protein complexes with a constant charge-to-size ratio.

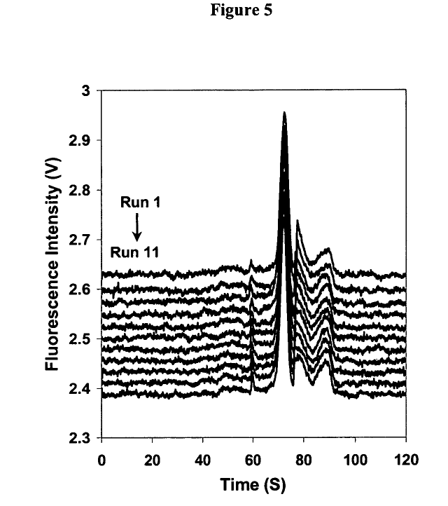

Protein separation using microchip electrophoresis in free solution was highly reproducible as shown in FIG. 5. In these experiments, electrophoresis was performed under an electric field of 500V/cm and 3 s injection time. The peaks were assigned as follows: 3-galactosidase—60 s; conalbumin—73 s; α-lactalbumin—78 s; and ovalbumin—90 s. Results indicate that the relative standard deviations of migration times from eleven consecutive runs were within 0.5% (0.37% (60 s), 0.28% (73 s), 0.27% (78 s), 0.43% (90 s)). There was no sign of serious adsorption of the proteins to the microchannel walls.

### OTHER EMBODIMENTS

While the invention has been described in conjunction with the detailed description, the foregoing description is intended to illustrate and not limit the scope of the invention, which is defined by the scope of the appended claims. Other aspects, advantages, and modifications are within the scope of the following claims.

What is claimed is:

1. A method for separating proteins comprising contacting proteins with a mixture of alkyl sulfates having different carbon chain lengths and separating the proteins in free solution using electrophoresis.

2. The method of claim 1, wherein the size of at least one protein is greater than about 10 kDa.

3. The method of claim 1, wherein the proteins are labeled.

4. The method of claim 3, wherein the proteins are labeled with a fluorogenic dye.

5. The method of claim 3, wherein the proteins are radioactively labeled.

6. The method of claim 5, wherein the proteins are labeled with S³⁵.

7. The method of claim 1, wherein the mixture of alkyl sulfates comprises an alkyl sulfate selected from the group consisting of dodecyl sulfate, tetradecyl sulfate, and hexadecyl sulfate.

8. The method of claim 1, wherein the mixture of alkyl sulfates comprises alkyl sulfates of about 2, 3, 4 or 5 different carbon chain lengths.

9. The method of claim 8, wherein the mixture of alkyl sulfates comprises two or three alkyl sulfates selected from the group consisting of dodecyl sulfate, tetradecyl sulfate, and hexadecyl sulfate.

10. The method of claim 1, further comprising heating the proteins and alkyl sulfates prior to separating the proteins.

11. The method of claim 10, wherein the proteins and alkyl sulfates are heated to about 95° C. for about 5 minutes.

12. The method of claim 10, wherein the proteins and alkyl sulfates are heated to about 80° C. to 95° C. for about 10 minutes.

13. The method of claim 10, wherein the proteins and alkyl sulfates are heated to about 50° C. to 80° C. for about 30 minutes.

14. The method of claim 1, wherein the proteins are in contact with a mixture of alkyl sulfates having different carbon chain lengths during the separation.

15. The method of claim 14, wherein the mixture of alkyl sulfates comprises alkyl sulfates of about 2, 3, 4 or 5 different carbon chain lengths.

16. The method of claim 15, wherein the mixture of alkyl sulfates comprises two or three alkyl sulfates selected from the group consisting of dodecyl sulfate, tetradecyl sulfate, and hexadecyl sulfate.

17. The method of claim 1, wherein the separation is performed on a microfluidic chip.

18. The method of claim 1, wherein the separation is performed in a capillary tube.

19. The method of claim 1, further comprising detecting at least one of the separated proteins.

20. The method of claim 19, wherein the proteins are detected using laser-induced fluorescence.

21. A method for isolating proteins, comprising:
a. obtaining a biological sample comprising proteins;
b. denaturing the proteins while the proteins are in contact with a mixture of alkyl sulfates having different carbon chain lengths; and
c. separating the proteins using electrophoresis, while the proteins are in contact with a mixture of alkyl sulfates having different carbon chain lengths.

22. The method of claim 21, further comprising labeling the proteins with a detectable marker.

23. A composition comprising one or more purified proteins and a mixture of alkyl sulfates having different carbon chain lengths, wherein the different carbon chain lengths are from about 3 to about 18 carbons, and wherein the composition comprises alkyl sulfates of about 2, 3 or 4 different carbon chain lengths.

24. The composition of claim 23, wherein the mixture of alkyl sulfates comprises two or three alkyl sulfates selected from the group consisting of dodecyl sulfate, tetradecyl sulfate, and hexadecyl sulfate.

25. The composition of claim 23, wherein the size of at least one of the purified proteins is greater than about 10 kDa.

26. The composition of claim 23 that has about 2 to about 10 different proteins.
